(19) United States
(12) Patent Application Publication
(10) Pub. No.: US 2018/0055563 A1
(43) Pub. Date: Mar. 1, 2018

(54) Solutions For Early Diagnoses, Prevention And Treatment Of Endometriosis

(71) Applicant: BOSTON SCIENTIFIC SCIMED, INC., Maple Grove, MN (US)

(72) Inventors: Jai SHETAKE, Santa Clarita, CA (US); Bryan Allen CLARK, Forest Lake, MN (US); Stephen B. RUBLE, Lino Lakes, MN (US); Timothy Paul HARRAH, Cambridge, MA (US); David J. TERNES, Roseville, MN (US)

(21) Appl. No.: 15/683,467

(22) Filed: Aug. 22, 2017

Related U.S. Application Data
(60) Provisional application No. 62/379,144, filed on Aug. 24, 2016.

(51) Int. Cl. A61B 18/14; A61N 1/05; A61N 1/36; A61N 1/40

(52) U.S. Cl. CPC A61B 18/1492; A61N 1/0521; A61B 2018/00023; A61N 1/36031; A61N 1/40; A61N 1/36007

(57) ABSTRACT

A system for treating abnormal muscle activity via energy application includes a catheter, an electrode assembly including a plurality of electrodes, a processor configured to receive signals associated with intrinsic tissue activity sensed by the plurality of electrodes, the processor determining a location of a target tissue to be treated based on the sensed intrinsic activity, and a stimulator configured to transmit energy to at least one of the plurality of electrode located at the target tissue.

### PRIORITY CLAIM

The present disclosure claims priority to U.S. Provisional Patent Application Ser. No. 62/379,144 filed Aug. 24, 2016; the disclosure of which is incorporated herewith by reference.

### BACKGROUND

Endometriosis (EM) is often misdiagnosed and untreated. Current interventions for EM include pharmacology and surgery. Pharmacologic interventions provide limited, short-term relief for pain associated with EM. Severe cases of EM are often treated with surgical options such as electric ablation of the endometrium or hysterectomies, which may have drastic side effects such as reduction or elimination of the chances of pregnancy.

### SUMMARY

The present disclosure relates to a catheter system comprising an elongated catheter body extending between a proximal end and a distal end, an electrode assembly coupled to the distal end of the catheter body, the electrode assembly comprising a plurality of arms, each of the arms including a plurality of electrodes, a processor configured to receive signals associated with intrinsic tissue activity sensed by the plurality of electrodes, the processor determining a location of a target tissue to be treated based on the sensed intrinsic tissue activity, and a stimulator coupled to the plurality of electrodes to deliver energy to the target tissue.

In an embodiment, the electrode assembly is in the form of a basket structure sized and dimensioned to fit within the uterus to treat endometriosis.

In an embodiment, the system further includes a display coupled to the processor configured to present real-time output of the electrodes.

In an embodiment, the system further includes a delivery sheath extending between a proximal end and a distal end, wherein the catheter and electrode assembly are slidably received within the delivery sheath, the electrode assembly sized and configured to fit in the sheath in a collapsed configuration.

In an embodiment, the electrode assembly includes a shape memory material that biases the arms toward an expanded configuration.

In an embodiment, the stimulator provides energy to the electrodes sufficient to block muscle activity.

In an embodiment, the system further includes a balloon coupled to the catheter and in fluid communication therewith.

In an embodiment, the system further includes a source of cooling fluid supplying cooling fluid to the balloon and withdrawing fluid from the balloon to remove heat from the target tissue to maintain a temperature of the target tissue below a predetermined threshold temperature.

In an embodiment, the system further includes a plurality of temperature sensors positioned at or between the plurality of electrodes to detect a temperature of the target tissue.

The present disclosure also relates to a system for treating endometriosis comprising an electrode assembly including a plurality of electrodes, a processor configured to receive signals associate with intrinsic tissue activity sensed by the plurality of electrodes, the processor determining a location of a target tissue to be treated based on the sensed intrinsic tissue activity, and an external stimulator configured to wirelessly transmit energy to at least one of the plurality of electrodes located at the target tissue.

In an embodiment, the electrode assembly comprises a plurality of arms, the plurality of electrodes positioned along the plurality of arms.

In an embodiment, the electrode assembly includes a shape memory material that biases the arms toward an expanded configuration.

In an embodiment, the plurality of electrodes is implanted directly into the target tissue.

In an embodiment, the external stimulator provides radiofrequency energy to the electrode assembly.

In an embodiment, the system further includes a delivery sheath extending between a proximal end and a distal end, the electrode assembly sized and configured to fit in the sheath.

The present disclosure also relates to method for treating endometriosis comprising positioning an electrode assembly within the uterus, the electrode assembly including a plurality of electrodes, sensing the intrinsic tissue activity with the plurality of electrodes, determining a location of a target tissue to be treated based on the sensed intrinsic tissue activity, and delivering energy to the target tissue via at least one of the plurality of electrodes.

In an embodiment, the method further includes delivering the electrode assembly to the uterus via a delivery sheath extending between a proximal end a distal end, the electrode assembly sized and shaped to fit within the sheath in a collapse configuration.

In an embodiment, the energy delivered is RF ablation energy.

In an embodiment, the electrode assembly is coupled to an elongated catheter body extending between a proximal end and a distal end.

In an embodiment, the method further includes supplying cooling fluid from a cooling fluid source to a balloon; and withdrawing the cooling fluid from the balloon to remove heat from the target tissue to maintain a temperature of the target tissue below a predetermined threshold temperature, wherein the balloon is coupled to the catheter and in fluid communication therewith.

### BRIEF DESCRIPTION

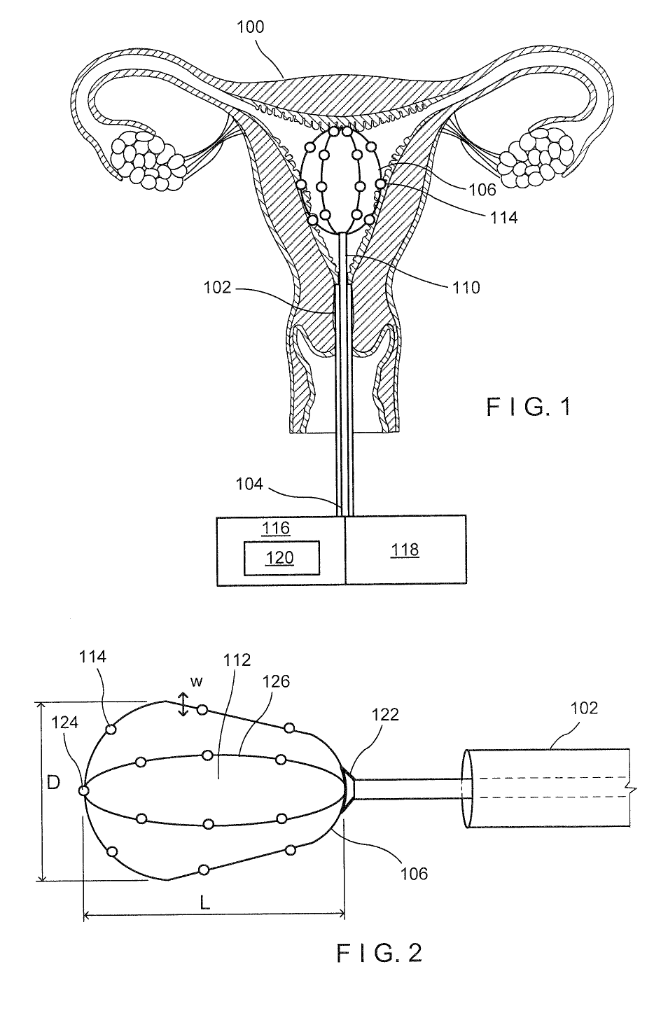

FIG. 1 shows a partially cross-sectional view of a device for mapping and electrically treating tissue according to an exemplary embodiment of the present disclosure;

FIG. 2 shows a partially cross-sectional view of an electrode structure of the device of FIG. 1 according to an exemplary embodiment of the present disclosure;

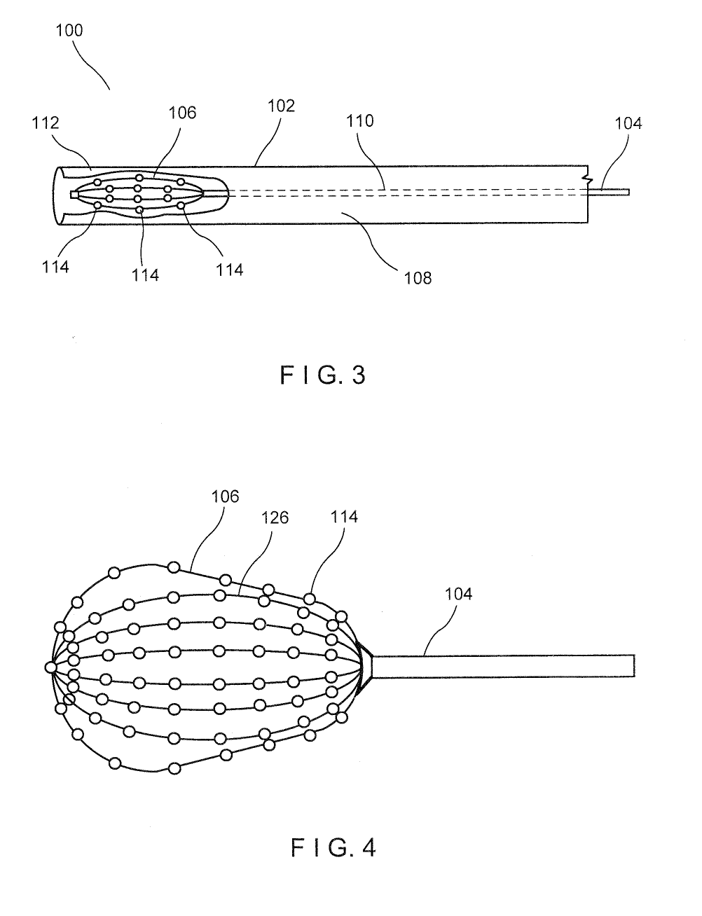

FIG. 3 shows another partially cross-sectional view of the electrode structure of FIG. 1 according to an exemplary embodiment of the present disclosure;

FIG. 4 shows a side view of an electrode structure according to a second embodiment of the present disclosure;

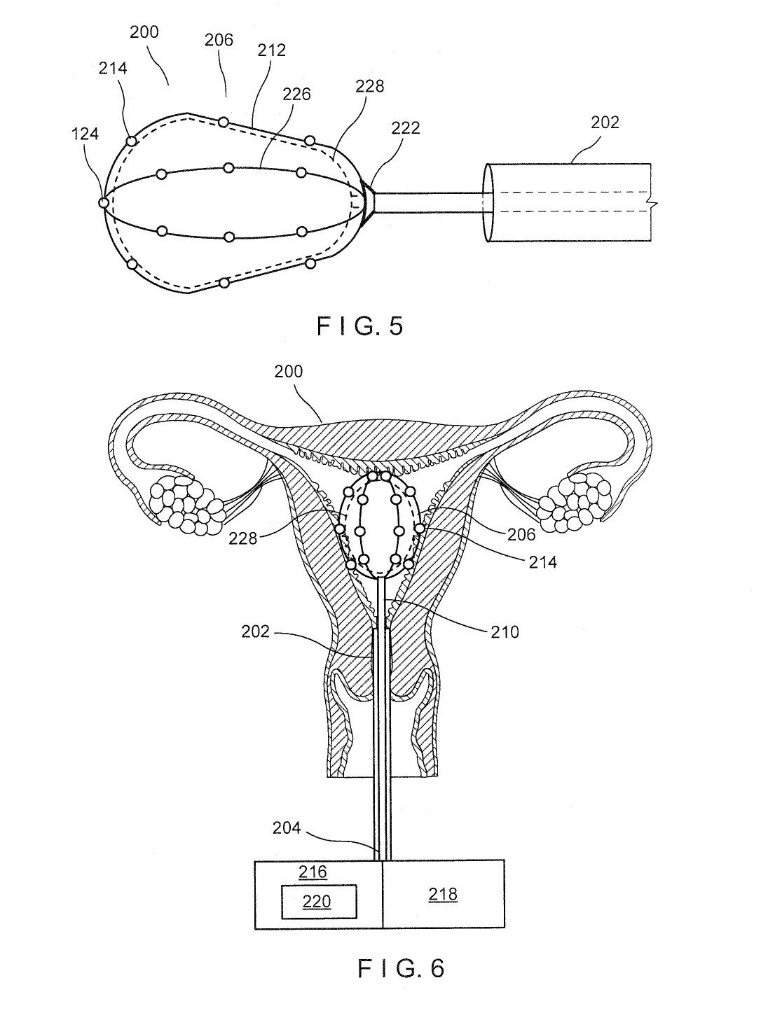

FIG. 5 shows a side view of an electrode structure according to a third embodiment of the present disclosure;

FIG. 6 shows a partially cross-sectional view of a device for mapping and electrically treating tissue according to the embodiment of FIG. 5;

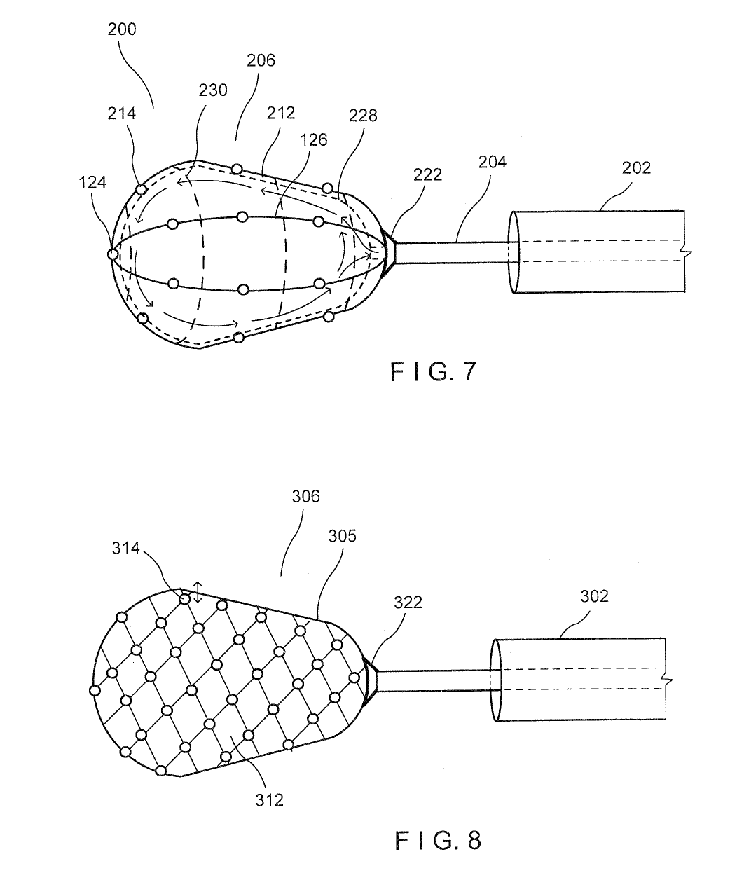

FIG. 7 shows a side view of an electrode structure according to a fourth embodiment of the present disclosure.

FIG. 8 shows a side view of an electrode structure according to a fifth embodiment of the present disclosure.

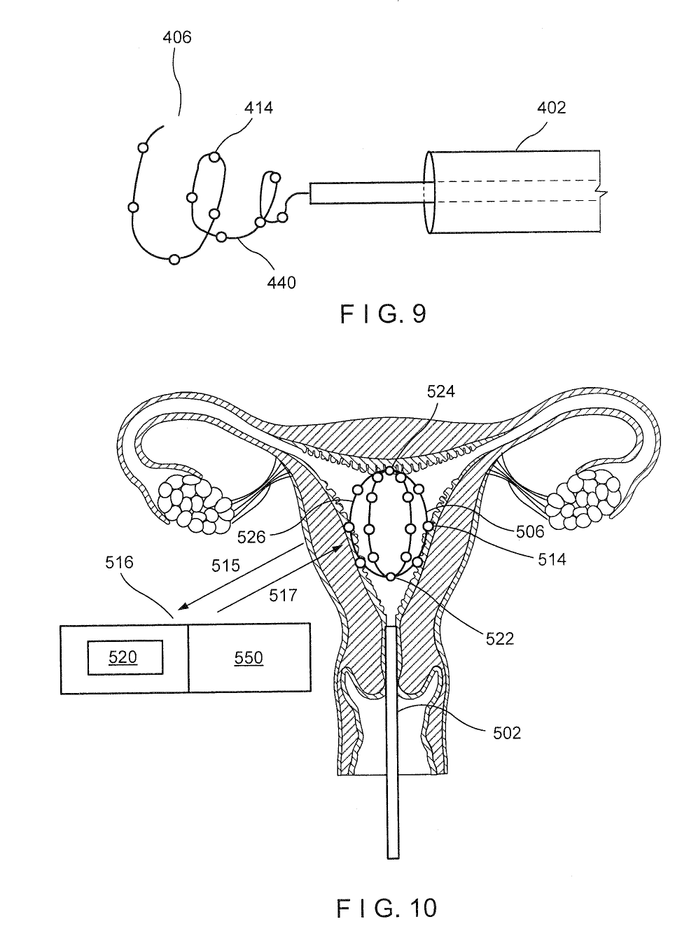

FIG. 9 shows a side view of an electrode structure according to a sixth embodiment of the present disclosure.

FIG. 10 shows a partially cross-sectional view of a device for mapping and electrically treating tissue according to another exemplary embodiment of the present disclosure; and

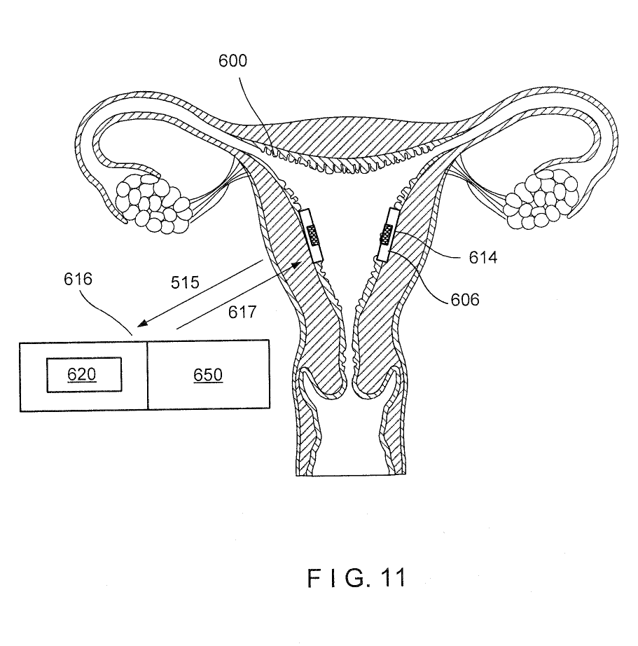

FIG. 11 shows a partially cross-sectional view of a device for mapping and electrically treating tissue according to another exemplary embodiment of the present disclosure.

### DETAILED DESCRIPTION

The present disclosure may be further understood with reference to the appended drawings and the following description, wherein like elements are referred to with the same reference numerals. The present disclosure relates to devices and methods for mapping and supplying energy to tissue and, more particularly, relates to catheter devices for mapping and supplying energy to tissue within the uterus. It should be noted that the terms “proximal” and “distal”, as used herein, are intended to refer to a direction toward (proximal) and away from (distal) a user of the device (e.g. physician).

As shown in FIGS. 1-3, a device 100 comprises an electrically insulative delivery element 102 within which a catheter 104 with a distal electrode structure 106 is slidably received. The delivery element 102 according to this embodiment may be sized, for example, to pass through the working channel of an endoscope or other insertion device for delivery to a target tissue within a living body. As would be understood by those skilled in the art, the device 100 is preferably sufficiently flexible to pass through a tortuous path through, for example, along a natural body lumen without undue trauma to tissue along and adjacent to the lumen or damage to the device 100 or a flexible endoscope or other insertion device. For example, the device 100 may have a flexibility sufficient to permit the device 100 to be slidably inserted through a working channel of a device such as a flexible endoscope and to pass through any bending radii that these devices might achieve. The delivery element 102 may be formed as a flexible sheath and defines an internal lumen 106 within which the basket catheter 104 is slidably received. In an exemplary embodiment, the delivery element is formed as a sheath of polyether ether ketone (PEEK) having an outer diameter of between 1 mm and 5 mm. However, as would be understood by those skilled in the art, other materials and sizes may be used.

The catheter 104 has a body 110 formed of a flexible material such as silicone, polyurethanes (PU), polytetrafluroethylene (PTFE), polyisobutylene polyurethane (PIB-PUR), poly(styrene-block-isobutylene-block-styrene) (SIBS), or any other suitable biocompatible flexible material. According to the exemplary embodiment, the catheter 104 may be formed, for example, having an outer diameter of less than 1 cm, preferable less than 5 mm. The catheter 104 of the exemplary embodiment extends between a proximal end which, in use, remains outside the body accessible to the user, and a distal end. The distal end of the catheter 104 includes a three-dimensional multiple electrode structure 106. In the illustrated embodiment, the structure 106 is formed as a basket defining an open interior space 112, although other multiple electrode structures could be used. The multiple electrode structure 106 is expandable from a reduced diameter insertion/withdrawal configuration in which the structure 106 fits within the catheter 104 and a deployed configuration in which the structure 106 is expanded radially to place electrodes 114 thereof in contact with tissue at locations dispersed about the uterine wall. It will be understood that the electrode structure 106 may expand in any suitable way such as, for example, via shape memory material, such as Nitinol, or an inflatable balloon disposed within the electrode structure 106. The electrodes 114 are configured in a known manner to sense intrinsic physiological activity in the anatomical region, which sensed activity is then processed by a processing/guidance system 116 to assist the physician in identifying a site or sites having abnormal uterine contraction patterns. This process is commonly referred to as mapping. As would be understood by those skilled in the art, this information can then be used to determine appropriate locations for applying therapy (e.g. electric stimulation or ablation) to the identified sites, and to navigate the one or more electrodes 114 to the identified sites.

Each of the electrodes 114 is individually electrically coupled to an external processing/guiding system 116. Those skilled in the art will understand that the electrodes 114 may, alternatively, be electrically connected to the external processing/guidance system 116 in any desired combinations and/or patterns. A lead wire (not shown) is electrically coupled to each electrode 114 on the electrode structure 106. The wires extend through the body 110 of the catheter 104 and electrically couple each electrode 114 to the processing/guiding system 116 independently of the other electrodes 114 so that signals sensed by each electrode 114 may be identified as such. Each of the electrodes 114 senses intrinsic electrical activity in a portion of the uterine wall with which the electrode 114 is in contact and this activity is processed by the processing/guiding system 116 to assist the physician in identifying, or mapping, a site or sites within the uterus appropriate for therapy delivery. The processing system 116 can process and provide position-specific information in various ways. It will be understood that target tissue to be treated may he determined by various other known methods in the art including, but not limited to, sensing local impedance or temperature, measuring local force compliance or measuring thickness of the uterus wall through ultrasound.

In an exemplary embodiment, once the electrode structure 106 identifies, or maps, the sites appropriate for therapy delivery and the processing system 116 provides position-specific information, a separate therapy delivery device (not shown) may be positioned at the known location to delivery therapy to the target tissue. In another exemplary embodiment, each electrode 114 is also individually electrically connected to a therapy delivery device 118 so that therapy may be provided to selected individual electrodes 114 to deliver therapy to the portions of tissue with which the selected electrodes 114 are in contact. It is understood that other forms of therapy such as, for example, microwave energy, ultrasound energy or radiofrequency energy, may be delivered to the electrodes 114. Once the electrode structure 106 has mapped/identified sites of uterine contractions, the processing system 116 identifies sites from which the abnormal contraction patterns originate. The therapy delivery device 118 may then stimulate the desired sites by delivering therapy to selected ones of the electrodes 114 to disrupt irregular uterine contraction activity in these areas and/or reversibly block sites where abnormal contraction patterns originate. Therapy may be delivered to any number of electrodes based on user input.

In the illustrated embodiment, the processing system 116 includes a user interface and output display device 120. The display device 120 presents the position-identifying output in real-time format most useful to the physician for remotely guiding the electrode structure 106 based on uterine contraction patterns. Additionally, the display device 120 may present other information useful to the physician for determining therapy parameters. For example, the display device 120 may display the magnitude of muscle activity from irregular contractions, which may be useful in assessing strength or duration of therapy to be delivered.

FIGS. 2-3 illustrate the electrode structure 106 in greater details. FIG. 2 shows the electrode structure 106 in an expanded profile, while FIG. 3 shows the electrode structure 106 in a collapsed profile inside the delivery element sheath 102. The illustrated electrode structure 106 comprises a base member 122 and an end cap 124 between which flexible splines 126 generally extend in a circumferentially spaced relationship. Each of the splines 126 has a distal and a proximal end. The base member 122 is configured as a lead-in feature to enable easy retraction of the electrode structure 106 back inside the delivery element sheath 102. In an embodiment, the end cap 124 may be another electrode. As discussed above, the electrode structure 106 takes the form of a basket defining an open interior space 112. The splines 126 are deflectable elongated pieces that carry electrodes 114 separated from one another along their lengths. In an exemplary embodiment, the electrode structure 106 is generally pear-shaped in a deployed configuration with a proximal portion having a smaller diameter than a distal portion to conform to the anatomy of the uterus, as can be seen in FIG. 2. In this embodiment, the electrode structure 106 may have, for example, a diameter of 2.5 cm, a length of 7.5 cm, and a width of 5 cm. It is understood, however, that the dimensions of the electrode structure 106 may vary depending on the optimal size necessary for specific anatomies and applications. In this embodiment, a plurality of electrodes 114 is disposed along the length of each of the splines 126, equidistant from one another. However, it is understood that the electrodes 114 may be disposed along the splines 126 in any manner or configuration. As illustrated, each spline 126 carries three electrodes 114. However, additional or fewer electrodes 114 could be disposed on each spline 126 in other embodiments of the electrode structure 106. For example, in another exemplary embodiment seen in FIG. 4, the electrode structure 106 may have a 64-electrode configuration where electrodes 114 are disposed in groups of eight electrodes 114 on each of eight splines 126. In an exemplary embodiment, the splines 126 are made of a biocompatible, resilient inert material such as, for example, Nitinol, and are connected between the base member 122 and the end cap 124 so that they may bend and conform to the tissue surface they contact. In the illustrated embodiment, four splines 124 form the electrode structure 106. Additional or fewer splines 124 could be used in other embodiments. Electrodes 114 may be affixed to the outer surface of the splines 124 in a suitable manner, including, e.g., adhesives, so that they can readily contact tissue when the assembly is expanded.

Delivery element sheath 102 is movable along the major axis of the catheter body 110. Moving the sheath 102 forward (i.e., toward the distal end) causes the sheath 102 to move over the catheter 110 and electrode structure 106, thereby collapsing the electrode structure 106 into a compact, low profile condition suitable for introduction into an interior space, such as, for example, into the cervix and uterus, as illustrated in FIG. 3. In contrast, moving the sheath 102 proximally frees the electrode structure 106, allowing the structure 106 to spring open and assume its basket-shaped structure illustrated in FIG. 2. Those skilled in the art will understand that the structure 106 may spring open when free from the constriction of the sheath 102 under a natural bias imparted to the material from which the structure 106 is formed. Alternatively, the structure 106 may be coupled to any known mechanism for mechanically expanding the structure 106 after it has been extended distally from the sheath 102. For example, the structure 106 may expand via shape memory material, such as Nitinol, or an inflatable balloon disposed within the electrode structure 106.

Each electrode 114 is electrically coupled to a wire (not shown) which extends through the body 110 of the catheter 104 and into a handle (not shown), in which the wires are coupled to an external connector (not shown). The connector electrically couples the electrodes 114 to the processing/guidance system 116 and therapy delivery device 118.

According to an exemplary method, delivery element 102, with the catheter 104 and electrode structure 106 disposed therein in the insertion configuration, is inserted through the cervix and into the uterus. Once the delivery element 102 is positioned within the uterus, it is moved proximally to deploy the electrode structure 106, which is free to expand its basket-shaped structure to the deployed configuration. In this configuration, the structure 106 is positioned adjacent to the anatomical structure to be treated (e.g. wall of the uterus) with the electrodes 114 in contact with the uterine wall. The processing/guidance system 116 then receives signals from the electrodes 114 relating to physiological activity of the tissue adjacent to each electrode 114 (e.g., uterine contraction patterns). That is, the electrodes 114 measure electrical impulses intrinsic to the physiological activity of the anatomical structures they contact.

Using the signals received from the electrodes 114, the processing/guiding system 116 is maps irregular contraction activity within the uterus and identifies regions to which therapy delivery is to be applied (e.g., to disrupt this irregular contraction activity). Based on the output generated by the processing/guiding system 116, the clinician may then decide to reposition the electrode structure 106 with respect to the uterus. For example, based on the output provided on the display device 120, the clinician may determine that the electrode structure 106 is positioned away from a target treatment site. When the clinician moves the electrode structure 106, the processing/guiding system 116 is configured and updated. When the electrode structure 106 is located in the desired position, therapy may be delivered to selected ones of the electrodes 114 in the regions from which irregular contraction activity is originating. Once therapy has been delivered to the electrodes 114 in the desired region, the intrinsic physiological activity of the uterus may be mapped again by the electrode structure 106 to confirm efficacy of the treatment.

As shown in FIGS. 5-7, a device 200 according to a further embodiment can be used to map physiological activity in a manner similar to that described above in regard to the device 100. However, instead of delivering therapy to the desired region to temporarily disrupt contractions as does the device 100, the device 200 applies an increased amount of therapy (e.g., radiofrequency RF energy) sufficient to ablate and irreversibly impair irregular muscle activity and/or irreversibly block sites from which abnormal contraction patterns originate. For example, with radiofrequency (RF) energy, therapy could be delivered to maintain target tissue temperature above at least about 60 degrees Celsius for a period of time sufficient to irreversibly impair the irregular muscle activity. In some examples, RF energy may be delivered in discrete activation of, e.g., five to ten seconds per activation. The frequencies of the RF energy may be from 300 to 1750 kHz, for example. It should be noted that, in at least some examples, other suitable values for energy delivery times, wattage, tissue temperature, and RF frequencies are also contemplated. In yet another example, with cryogenic therapy, the tissue temperature may be maintained in the range of about −5 to −50 degrees Celsius for a period of time sufficient to irreversibly impair the irregular activity. The device 200 includes an electrically insulative, flexible delivery element 202 which may be substantially the same as the delivery element 102 of the device 100 with a catheter 204 and distal electrode structure 206 slidably received therein. The catheter 204 and electrode structure 206 may be formed of the same materials and in the same or different dimensions as described above for the catheter 104 and electrode structure 106 as may be dictated by the procedure for which the device is to be used.

The catheter 204 of this embodiment extends between a proximal end which, in use, remains outside the body accessible to the user, and a distal end. Electrode structure 206 is located at the distal end of the catheter 204 and is movable between a first insertion position in which the sheath of the delivery element 202 is moved distally, causing the electrode structure 206 to be held within the sheath of the delivery element 202, and a second position in which the sheath of the delivery element 202 is moved proximally, allowing the electrode structure 206 to spring open and assume its basket-shaped structure.

The electrode structure 206 of this exemplary embodiment extends between a proximal end comprising base member 222 and a distal end. As with electrode structure 106, electrode structure 206 may take the form of a basket defining an open interior space 212. The electrode structure 206 carries a plurality of electrodes 214 configured to sense intrinsic physiological activity in the anatomical region on which the therapy delivery is to be performed. However, in this exemplary embodiment, the electrode structure 206 may include a balloon 228 positioned within the structure 206 to aid in expansion of the electrode structure 206 for intimate contact with the uterus wall. The balloon 228 may be free-floating, for example, with a basket electrode structure 206 surrounding the balloon 228. In another example, the balloon 228 may be adhered to parts of the structure 206 (via e.g., adhesives, applied directly to an outer surface of the balloon 228). The balloon 228 may be inflated with air or a fluid (e.g. sterile saline). According to an exemplary embodiment, the balloon 228 and electrode structure 206 are generally pear-shaped in a deployed configuration, with a proximal portion of each having a smaller diameter than distal portions thereof. In an exemplary embodiment, the balloon 228 with electrode structure 206 may have, for example, in the deployed configuration an outer diameter of 2.5 cm, a length of 7.5 cm and a width of 5 cm.

The electrodes 214 are individually electrically coupled to a processing/guiding system 216 similar to the processing/guiding system 116. A guide wire (not shown) is electrically coupled to each electrode 214 on the electrode structure 206. The wires extend through the body 210 of the catheter 204 and electrically couple the electrodes 214 to the processing/guiding system 216. The electrodes 214 sense intrinsic electrical activity in the uterus tissue. The sensed activity is processed by the processing system 216 to assist the physician in identifying, or mapping, the site or sites within the uterus appropriate for ablation. The processing system 216 can process and provide position-specific information in various ways. In this exemplary embodiment, the electrodes 214 are also electrically connected to a therapy delivery device 218 configured to deliver therapy to the one or more electrodes 214. Once the electrode structure 206 maps/identifies sites of uterine contractions, the processing/guiding system 216 identifies sites from which abnormal contraction patterns originate. The therapy delivery device 218 may then ablate target sites selected in a pattern designed to permanently disrupt this irregular contraction activity (e.g., by isolating the origination sites to prevent the propagation of activity therefrom) thereby regulating uterine contractions.

In an exemplary embodiment, one or both of the electrode structure 206 and the balloon 228 have temperature sensors (e.g. thermistors) 230 positioned at or between the electrodes 214 to monitor ablation therapy delivery, as shown in FIG. 7. Temperature sensors 230 may monitor the temperature of the tissue being treated to avoid scorching and/or charring of the surface tissue during treatment as this impedes the delivery of energy to tissue beyond this scorched or charred tissue. Temperature sensors 230 may be coupled or adhered to the electrode structure 206 in any suitable manner. In another embodiment, the temperature sensors 230 may be adhered only to the balloon 228. Temperature sensors 230 may be electrically coupled to the external processing system 216. The processing system 216 can then process and provide position-specific temperatures to allow the clinician to monitor tissue treatment and/or to facilitate the control of the temperature to which treated tissue is raised (e.g., to avoid charring) either automatically or manually under user control.

In the illustrated embodiment, the processing/guiding system 216 includes a user interface and output display device 220. The display device 220 presents the position-identifying output in real-time format most useful to the physician for remotely guiding the electrode structure 206 based on uterine contraction patterns.

During use, cooling fluid (e.g. sterile saline) may be supplied to the electrode structure 206 or balloon 228 from an external supply source via catheter 204 to maintain a temperature of the portion below a threshold level at which charring occurs, as shown in FIG. 7. For example, the threshold for irreversible effect to tissue may be above 45 degrees Celsius or, more particularly, above 60 degrees Celsius. The cooling flow rate through the catheter is sufficient to maintain the temperature at the interface between the electrode(s) and the tissue at or below the temperature of target tissue away from the electrode(s). For example, the electrode temperature may be maintained below 45 degrees Celsius while the target tissue temperature is at a temperature of approximately 60 degrees Celsius, in one configuration, with a cooling fluid flow rate of approximately 0.5 mL per second. In at least one example, one or more temperature sensors in at least one electrode may be used to control energy delivery to maintain electrode temperature below a defined threshold to avoid undesirable charring. By preventing the charring of surrounding tissue, the electrode structure 206 according to this embodiment can achieve desired effects on deeper muscle/nerve tissue as energy is transferred more efficiently through tissue that has not been charred. This permits the system 200 to disrupt irregular activity more effectively in cases where the irregular contraction activity has origins in deeper tissue. In an exemplary embodiment, cooling fluid may be provided through the catheter 204 and circulated through the catheter 204 and/or balloon 228 (movement of cooling fluid is depicted by arrows in FIG. 7). In another embodiment, fluid may be supplied by the catheter 204 into the electrode structure 206. In this embodiment, electrode structure 206 may have pores or holes 232 to allow fluid to exit the electrode structure 206 and directly cool the ablated tissue externally.

According to an exemplary method, delivery element 202, with the catheter 202, electrode structure 206 and balloon 228 disposed therein in the insertion configuration, is inserted through the cervix and into the uterus (e.g., via an insertion device). Once the delivery element 202 is positioned as desired within the uterus, the electrode structure 206 and the balloon 228 are deployed distally from the catheter 202. The balloon 228 is then inflated with a biocompatible fluid to expand the electrode structure 206 which is urged radially outward by the balloon 228 into its deployed configuration with the basket-shaped structure 206 in contact with the uterine walls. After the balloon 228 and electrode structure 206 have been positioned adjacent to the anatomical structure to be treated (e.g. wall of the uterus) as desired, the processing/guidance system 216 receives signals from the electrodes 214 related to intrinsic physiological activity of adjacent anatomical structures (i.e. uterine contraction patterns). That is, the electrodes 214 measure electrical impulses intrinsic to the physiology of the anatomical structures with which they are in contact.

Using the signals received from the electrodes 214, the processing/guiding system 216 maps irregular activity within the uterus and identifies regions to which electrical energy should be applied to disrupt the irregular activity. Based on the output generated by the processing/guiding system 216, the clinician then repositions the electrode structure 206 with respect to the uterus if needed to position the electrodes 214 as desired relative to the regions to which therapy is to be applied. For example, based on the output provided on the display device 220, the clinician may determine that the electrodes 214 are spaced away from one or more target treatment sites. When the clinician moves the electrode structure 206, the processing/guiding system 216 is reconfigured and updated until the electrode structure 206 is located in a desired position optimized for application of therapy to target treatment sites. The clinician is also able to select which electrodes therapy is delivered to as well, thus eliminating or reducing the need to reposition the electrode structure 206. Therapy is then delivered to the electrodes 214 positioned adjacent to the target treatment sites (e.g., regions from which irregular contraction activity originates) via the therapy delivery device 218 to impair irregular contraction patterns within the uterus. In an exemplary method, ablation lines or rings may be created between contracting muscles and sites from which signals propagating these irregular contractions originate or along the muscles themselves to prevent these muscles from contracting. Additionally, or alternatively, nerves or neuromuscular junction may be impaired to reduce neural input to the muscles and similarly reducing irregular muscle contractions. This may be accomplished by maintaining the target tissue temperature above, for example, 60 degrees Celsius for a period of time sufficient to irreversibly damage the target tissue. Alternatively, it may be accomplished by maintaining the target tissue temperature below approximately −5 degrees Celsius for a period of time sufficient to irreversibly damage the target tissue. While the ablation energy is being delivered, cooling fluid 232 may be circulated through the balloon 228 and/or electrode structure 206 to maintain the temperature of the surface tissue below a level at which charring occurs to permit ablation of deeper muscle/nerve tissue. For example, temperature sensors 230 may provide real-time feedback of the temperature of tissue being treated to inform the clinician whether the target tissue is reaching a threshold (e.g., the charring temperature). Once an amount of therapy delivered from the electrodes 214 to the desired regions is sufficient to ablate the tissue as desired, the intrinsic physiological activity of the uterus may be mapped again by the electrode structure 2106 to confirm efficacy of the treatment. The process may then be repeated as necessary until the desired effect has been achieved.

FIGS. 8 show various alternate configurations of electrode structure 306 for mapping and energy delivery devices similar to devices 100 and 200. These devices differ only in the construction of the electrode structure 306 but are otherwise internally and operationally similar. The electrode structure 306 of FIG. 8 is a self-expanding stent 305 formed of, for example, Nitinol or any other known biocompatible, shape memory material. FIG. 8 shows the stent 305 in an expanded profile. The illustrated stent 306 comprises a lead in feature 322 at a proximal end to retract the stent 306 inside the sheath 302. The stent 305, when expanded, takes the form of a basket defining an open interior space 312. In an exemplary embodiment, the stent 305 is generally pear-shaped in a deployed position, with a proximal portion having a smaller diameter than a distal portion to conform to the anatomy of the uterus. In this embodiment, the stent 305 may have, for example, a diameter of 2.5 cm, a length of 7.5 cm, and a width of 5 cm. It is understood, however, that the dimensions of the stent 305 may vary depending on the optimal size necessary for specific anatomies and applications. In this embodiment, a plurality of electrodes 314 is disposed about the outer circumference of the stent 305. In an exemplary embodiment, the electrodes 314 are disposed about the stent 305 at points equidistant from one another. However, it is understood that the electrodes 314 may be located about the stunt 305 in any manner or configuration. Electrodes 314 may be affixed to the outer surface of the stent 305 in a suitable manner, including, e.g., adhesives, so that they can readily contact tissue when the assembly is expanded.

As shown in FIG. 9, the electrode structure 406 is configured as a spiral, self-expanding structure formed of, for example, Nitinol or any other biocompatible, shape memory material. The illustrated electrode structure 406 includes a flexible planar member or band 440 resembling a “ribbon” extending from a proximal end to a distal end in a generally spiral configuration. In an exemplary embodiment, the electrode structure 406 moves from a neutral or contracted configuration in which the “ribbon” 440 is not spiral-shaped, but rather extends linearly within the delivery element lumen 408 while the delivery element 402 is moved through a patient's anatomy toward the target location and before it is deployed from the delivery element 402 into the uterus. When the delivery element 402 is moved proximally to expose the structure 406, the electrode structure 406 expands into its spiral, deployed configuration in which the electrodes 414 contact the uterine wall as described above in regard to the devices 100, 200 and 300. In another embodiment, the electrode structure 406 retains its spiral configuration but in a contracted form when held within delivery element lumen 408 and simply expands radially when the delivery element 402 is retracted proximally to expose the structure 406. The ribbon 440 may be constructed of any suitable material as described above. The length of the ribbon electrode structure 406 may between approximately 5 cm and 30 cm in its linear configuration, for example. It is understood that the ribbon 440 may be constructed as a solid member, a woven member or a web member, provided the structure is sufficiently rigid yet flexible to support the electrodes 414 and hold the generally spiral form in the expanded and contracted configurations. A plurality of electrodes 414 extend along the ribbon electrode structure 406 from a proximal end to a distal end. In the illustrated embodiment, electrodes 414 are evenly spaced and span nearly the length of the ribbon 440 along an outer surface of the electrode structure 406. Electrodes 414 may be affixed to the outer surface of the electrode structure 406 in a suitable manner, including, e.g. adhesives, so that they can readily contact tissue when the assembly is expanded. Each electrode 414 is connected to a respective lead wire (not shown). In the illustrated embodiment, lead wires travel alongside the ribbon 440 and into the body of the catheter 404. Lead wires may be insulated as the traverse alongside the catheter body from the electrodes to the proximal end of the device 400. Insulation may be accomplished via individual insulative coverings over each lead wire and/or a multi-lumen insulative tubing serving as the catheter body through which the lead wires pass. Exemplary insulative materials include polyurethane (PU), silicone, ETFE and any other suitable insulative material.

As shown in FIG. 10, a device 500 according to a further embodiment can be used to map physiological activity and deliver electric energy in a manner similar to that described above in regard to the device 100. The device 500 includes an electrically insulative, flexible delivery element 502 which may be substantially the same as the delivery element 102 of the device 100 with electrode structure 506 slidably received therein. In this embodiment, however, the electrode structure 506 is not coupled to a catheter but rather, is a free-standing electrode structure 506 that is permanently or semi-permanently implantable within the uterus of the patient. The electrode structure 506 may be formed of the same materials and in the same or different dimensions as described above for electrode structure 106 as may be dictated by the procedure for which the device is to be used.

The illustrated electrode structure 506 comprises a base member 522 and an end cap 524 between which flexible splines 526 generally extend in a circumferentially spaced relationship similar to electrode structure 106. Base member 522 and end cap 524, in an exemplary embodiment, may be electrodes. As with electrode structure 106, electrode structure 506 is in the form of a basket defining an open interior space 512. The electrode structure 506 is movable between a first insertion position within the delivery element sheath 502 and a second position in which the sheath 502 is moved proximally to expose the electrode structure 506 permitting the structure 506 to spring open and assume its basket shaped in a manner similar to the electrode structure 106. The electrode structure 506 carries a plurality of electrodes 514 configured to sense intrinsic physiological activity in the anatomical region on which the therapy is to be performed However, in this exemplary embodiment, rather than each electrode 514 being electrically coupled to a processor/guiding system, the electrodes 514 of the electrode structure 506 sense intrinsic electrical activity in the uterus tissue and transmit any information about the activity wirelessly to an external processing device 516. This processing device 516 then processes this data and provides position-specific information in the same manner described above. In an exemplary embodiment, the wireless processing device 516 is wearable and alerts the patient to activate therapy when necessary. For example, once the electrode structure 506 maps/identifies sites of uterine contraction, the processing system may identify sites from which abnormal contraction patterns originate and monitors these sites during and after therapy delivery. An external radiofrequency (RF) generator 550 transmits RF energy 517 to the electrodes 514, which are configured to receive and deliver RF energy 517 wirelessly. In an exemplary embodiment, RF generator 550 is housed within the wireless device 516 to allow for easy transport with the patient.

According to an exemplary method, delivery element 502, with electrode structure 506 disposed therein in the insertion configuration, is inserted through the cervix and into the uterus. Once the delivery element 502 is positioned within the uterus as desired, it is withdrawn proximally to expose and deploy the electrode structure 506 which, when free from the constraint of the delivery element 502, assume its basket shape with the electrodes 514 in contact with target portions of the uterine wall. The electrode structure 506, which is no longer connected to any other element, is held in place via splines 526 which are configured to match a shape of the uterus walls. The electrode structure 506 is positioned within the anatomical structure to be treated (e.g., the uterus) and the processing system 516 receives signals 515 from the electrodes 514 related to intrinsic physiological activity of the anatomical structure (i.e., uterine contraction patterns). Using the signals received from the electrodes 514, the wireless processing device 516 then maps irregular activity within the uterus and identifies regions to which therapy is to be delivered to control the irregular activity. Output may be provided on a display 520 of the wireless processing device 516. Therapy may be delivered in amounts designed to reversibly or permanently block altered muscle activity as desired. Because, in this exemplary embodiment, the processing system 516 and RF generator 550 are portable, the processor 516 may alert the patient to activate therapy whenever the electrode structure 506 senses that irregular uterine activity is detected. The patient is then able to activate the RF generator 550 to transmit RF energy to the electrode structure 506 to block altered muscle activity as desired.

As shown in FIG. 11, a device 600 according to a further embodiment can be used to map physiological activity and deliver therapy in a manner similar to that described above in regard to the devices 100 and 500. The device 600 includes an electrically insulative, flexible delivery element 602 which may be substantially the same as the delivery element 102 of the device 100 with electrode structure 606 slidably received therein. In this embodiment, however, one or more free-standing electrode structures 606 are permanently or semi-permanently implantable within the lining or wall of the uterus of the patient. The electrode structures 606 may be formed of the same materials and in the same or different dimensions as described above for electrode structure 106 as may be dictated by the procedure for which the device is to be used.

The illustrated electrode structures 606 are configured as small stimulation units comprising individual electrodes 614 with small inductive coils for RF transmission. In an exemplary embodiment, electrode structures 606 may be substantially cylindrical and have, for example, an outer diameter of less than 5 mm and a length of less than 2 cm. In this exemplary embodiment, as with electrode structure 506, rather than each electrode 614 being electrically coupled to processor/guiding system, the electrodes 614 of the electrode structure 606 sense intrinsic electrical activity in the uterus tissue and transmit any information about the activity to a wireless processing device 616. This wireless processing device 616 can then process and provide position-specific information in various ways. In an exemplary embodiment, the wireless processing device 616 is wearable and alerts the patient to activate therapy when necessary. For example, once the electrode structure 606 maps/identifies sites of uterine contraction, the processing system may identify sites where abnormal contraction patterns originate and monitor these sites during and after energy application. An external radiofrequency (RF) generator 650 transmits RF energy to the electrodes 614, which are configured to receive and deliver RF energy wirelessly. In an exemplary therapy setting, up to approximately 30 mA current could be delivered to one or more electrodes at a frequency of up to 10 kilohertz with a pulse width of up to 500 microseconds. These settings are meant to be exemplary only. In general, with higher frequencies, it is expected that a blocking effect may be accomplished. At lower frequencies, for example, less than 100 hertz, activation of nervous or muscle tissue may alternatively be accomplished. In an exemplary embodiment, RF generator 650 is housed within the wireless device 616 to allow for easy transportation with the patient.

According to an exemplary method, delivery element 602, with electrode structure 606 disposed therein in the insertion configuration, is inserted through the cervix and into the uterus. Once the delivery element 602 is positioned within the uterus, it is moved proximally to deploy the electrode structure 606. Electrode structure 606 is positioned within the anatomical structure to be treated (e.g. the uterus) and the processing system 616 receives signals 615 from the electrodes 614 related to intrinsic physiological activity of the anatomical structure (i.e. uterine contraction patterns). Using the signals received from the electrodes 614, the wireless processing device 616 is then able to map irregular activity within the uterus and identify which regions to apply electrical energy. Based on the output provided on a display 620 on the wireless processing device 616, therapy may be delivered to the electrodes 614 in the regions of irregular contraction activity via the wireless RF generator 650 to reversibly block altered muscle activity. Because, in this exemplary embodiment, the processing system 616 and RF generator 650 are portable, the processor 616 may alert the patient to activate therapy whenever the electrode structure 606 senses that irregular uterine activity is detected. The patient is then able to activate the RF generator 650 to transmit RF energy 617 to the electrode structure 606 to block altered muscle activity when necessary.

In another exemplary embodiment, power may be self-contained inside a stimulation unit 618 within the electrode structure 606. In this embodiment, once irregular uterine activity is detected by the electrodes within the electrode structure 606, the stimulation unit 618 automatically provides therapy to the target tissue to block the detected altered muscle activity.

It will be apparent to those skilled in the art that various modifications may be made in the present disclosure without departing from the scope of the disclosure. Thus, it is intended that the present disclosure cover modifications and variations of this disclosure provided that they come within the scope of the appended claims and their equivalents.

1-15. (canceled)

16. A catheter system, comprising:
an elongated catheter body extending between a proximal end and a distal end;
an electrode assembly coupled to the distal end of the catheter body, the electrode assembly comprising a plurality of arms, each of the arms including a plurality of electrodes;
a processor configured to receive signals associated with intrinsic tissue activity sensed by the plurality of electrodes, the processor determining a location of a target tissue to be treated based on the sensed intrinsic tissue activity; and
a stimulator coupled to the plurality of electrodes to deliver energy to the target tissue.

17. The system of claim 16, wherein the electrode assembly is in the form of a basket structure sized and dimensioned to fit within the uterus to treat endometriosis.

18. The system of claim 16, further comprising:
a display coupled to the processor configured to present real-time output of the electrodes.

19. The system of claim 16, further comprising:
a delivery sheath extending between a proximal end and a distal end, wherein the catheter and electrode assembly are slidably received within the delivery sheath, the electrode assembly sized and configured to fit in the sheath in a collapsed configuration.

20. The system of claim 16, wherein the electrode assembly includes a shape memory material that biases the arms toward an expanded configuration.

21. The system of claim 16, wherein the stimulator provides energy to the electrodes sufficient to block muscle activity.

22. The system of claim 21, further comprising:
a balloon coupled to the catheter and in fluid communication therewith.

23. The system of claim 22, further comprising:
a source of cooling fluid supplying cooling fluid to the balloon and withdrawing fluid from the balloon to remove heat from the target tissue to maintain a temperature of the target tissue below a predetermined threshold temperature.

24. The system of claim 21, further comprising:
a plurality of temperature sensors positioned at or between the plurality of electrodes to detect a temperature of the target tissue.

25. A system for treating endometriosis, comprising:
an electrode assembly including a plurality of electrodes;
a processor configured to receive signals associate with intrinsic tissue activity sensed by the plurality of electrodes, the processor determining a location of a target tissue to be treated based on the sensed intrinsic tissue activity; and
an external stimulator configured to wirelessly transmit energy to at least one of the plurality of electrodes located at the target tissue.

26. The system of claim 25, wherein the electrode assembly comprises a plurality of arms, the plurality of electrodes positioned along the plurality of arms.

27. The system of claim 25, wherein the electrode assembly includes a shape memory material that biases the arms toward an expanded configuration.

28. The system of claim 25, wherein the plurality of electrodes is implanted directly into the target tissue.

29. The system of claim 25, wherein the external stimulator provides radiofrequency energy to the electrode assembly.

30. The system of claim 25, further comprising:
a delivery sheath extending between a proximal end and a distal end, the electrode assembly sized and configured to fit in the sheath.

31. A method for treating endometriosis, comprising:
positioning an electrode assembly within the uterus, the electrode assembly including a plurality of electrodes;
sensing the intrinsic tissue activity with the plurality of electrodes;
determining a location of a target tissue to be treated based on the sensed intrinsic tissue activity; and
delivering energy to the target tissue via at least one of the plurality of electrodes.

32. The method of claim 31, further comprising:
delivering the electrode assembly to the uterus via a delivery sheath extending between a proximal end a distal end, the electrode assembly sized and shaped to fit within the sheath in a collapse configuration.

33. The method of claim 31, wherein the energy delivered is RF ablation energy.

34. The method of claim 33, wherein the electrode assembly is coupled to an elongated catheter body extending between a proximal end and a distal end.

35. The method of claim 34, further comprising:
supplying cooling fluid from a cooling fluid source to a balloon; and
withdrawing the cooling fluid from the balloon to remove heat from the target tissue to maintain a temperature of the target tissue below a predetermined threshold temperature, wherein the balloon is coupled to the catheter and in fluid communication therewith.
